(19) United States
(12) Patent Application Publication
(10) Pub. No.: US 2002/0048580 A1
(43) Pub. Date: Apr. 25, 2002

(54) Method of inducing lung branching

(75) Inventors: Richard Mollard, Victoria (AU); Pierre Chambon, Blaesheim (FR); Manuel Mark, Morchwiller (FR)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Institut National De La Sante Et De La Recherche Medicale

(21) Appl. No.: 09/874,993

(22) Filed: Jun. 7, 2001

Related U.S. Application Data
(60) Provisional application No. 60/209,849, filed on Jun. 7, 2000.

(51) Int. Cl. A61K 39/395; A61K 31/07

(52) U.S. Cl. 424/145.1; 514/725

(57) ABSTRACT

The invention relates to a method of treating lung branching malformation in a subject by administering to the subject a pharmaceutically effective amount of a retinoic acid receptor (RAR) retinoid. The invention also relates to a method of increasing alveoli in a subject by administering a pharmaceutically effective amount of a retinoic acid receptor (RAR) antagonist. The invention further relates to a method of inducing primary lung bud formation in a subject by administering a pharmaceutically effective amount of a retinoic acid receptor agonist. The invention also relates to a method of identifying an agent capable of inducing primary lung bud formation, the method comprising: (a) administering an agent to an embryo; and (b) determining primary lung bud formation of said embryo.

This application claims benefit of the earlier filing date of U.S. Appl. No. 60/209,849, filed Jun. 7, 2000, the content of which is herein incorporated by reference in its entirety.

### BACKGROUND OF THE INVENTION

1. Field of the Invention

The invention relates to methods of treating lung branching malformation in a subject by administering to the subject a pharmaceutically effective amount of a retinoic acid receptor (RAR) retinoid. The invention also relates to methods of increasing alveoli in a subject by administering a pharmaceutically effective amount of a retinoic acid receptor (RAR) antagonist. The invention further relates to methods of inducing primary lung bud formation in a subject by administering a pharmaceutically effective amount of a retinoic acid receptor agonist. The invention also relates to methods of identifying an agent capable of inducing primary lung bud formation, the method comprising: (a) administering an agent to an embryo; and (b) determining primary lung bud formation of said embryo.

2. Background Art

Vitamin A (retinol) exerts multiple effects upon vertebrate development through binding of active metabolites, retinoic acids (RA), to two families of nuclear receptors, the retinoic acid receptors (RAR isotypes α, β and γ and their isoforms) and the retinoid X receptors (RXR isotypes α, β and γ and their isoforms) (reviewed in Kastner, P., et al., Cell 83: 859-869 (1995); and Chambon, P., FASEB. J. 10:940-954 (1996)). RARs bind all-trans RA (t-RA) and 9-cis RA (9c-RA), whereas RXRs bind only 9c-RA. RAR/RXR heterodimers regulate transcription of RA target genes through their activation function domains (AF1 and AF2) and their binding to conserved cis-acting RA response elements (RARβs; reviewed in Mangelsdorf, D. J., et al., Cell 83:835-839 (1995); Chambon, P., FASEB. J. 10:940-954 (1996)). Retinoid metabolism might be controlled by cytoplasmic binding proteins such as the cellular retinol binding proteins (CRBPI and II) and cellular retinoic acid binding proteins (CRABPI and II) (Napoli, J. L., Biochim. Biophys. Acta 1440:139-162 (1999)).

Lung development includes: (i) evagination of the primitive lung and tracheal buds from the embryonic foregut, (ii) branching morphogenesis, which essentially takes place during the pseudoglandular stage and establishes the primitive conducting airways and presumptive terminal sacs, and (iii) alveologenesis, characterized by septation of the terminal sacs to form definitive alveoli (Hogan, B. L. and Yingling, J. M., Curr. Opin. Genet. Dev. 8:481-486 (1998) and references therein). The importance of RA signaling for prenatal lung development was first established with the finding that vitamin A-deficient (VAD) rat fetuses often display, among other malformations, severe bilateral lung hypoplasia, lung agenesis and agenesis of the oesophagotracheal septum (Warkany J., et al., Pediatrics 1:462-471 (1948); Wilson, J. G., et al., Am. J. Anat. 92:189-217 (1953)). VAD-related congenital lung and tracheal malformations are also observed in RARα^{-/-}/RARβ⁻, RARα^{-/-}/RARβ2^{-/-}, RARα^{-/-}/RXRα^{-/-} and RARα^{-/-}/RXRαAF2° fetuses (Lohnes, D., et al, Development 120:2723-2748 (1994); Mendelsohn, C., et al., Development 120:2749-2771 (1994); Kastner, P., et al., Cell 78:987-1003 (1994); Kastner, P., et al., Development 124:313-326 (1997); Ghyselinck, N. B., et al., Int. J. Dev. Biol. 41:425-447 (1997); Mascrez, B., et al., Development 125:4691-4707 (1998)).

The addition of RA to growth-arrested pre-natal lung explants provided evidence that RA signaling could be involved in the stimulation of lung branching (Schuger, L., et al., Dev. Biol. 159:462-473 (1993)). However, independent studies have shown an inhibitory effect of RA treatment upon branching morphogenesis in lung cultures (Cardoso, W. V., et al., Am. J. Respir. Cell Mol. Biol. 12:464-476 (1995)). Postnatal treatment with retinoic acid has been shown to increase pulmonary alveoli in rats (Massaro, G. D. and Massaro, D., Am. J. Physio. 270: L305-L310 (1996); Massaro, G. D. and Massaro, D., Nat. Med. 3:675-677 (1997); U.S. Pat. No. 5,998,486). The role of retinoids in lung development is reviewed in Chytil, F., FASEB J. 10:986-992 (1996).

### BRIEF SUMMARY OF THE INVENTION

The invention is directed to a method of treating lung branching malformation in a subject by administering to the subject a pharmaceutically effective amount of a retinoic acid receptor (RAR) retinoid. The invention is also directed to a method of increasing alveoli in a subject by administering a pharmaceutically effective amount of a retinoic acid receptor (RAR) antagonist. The invention is further directed to a method of inducing primary lung bud formation in a subject by administering a pharmaceutically effective amount of a retinoic acid receptor agonist. The invention is also directed to a method of identifying an agent capable of inducing primary lung bud formation, the method comprising: (a) administering an agent to an embryo; and (b) determining primary lung bud formation of said embryo.

### BRIEF DESCRIPTION OF THE FIGURES

FIG. 1. Representative transverse histological sections through the primary left and right lung buds of three E8.0 embryos cultured for 48 hours.

FIG. 1A. Transverse histological section through the primary left and right lung buds of three E8.0 embryos cultured for 48 hours in vehicle (ethanol) alone.

FIG. 1B. Transverse histological section through the primary left and right lung buds of three E8.0 embryos cultured for 48 hours in vehicle (ethanol) alone.

FIG. 1C. Transverse histological section through the primary left and right lung buds of three E8.0 embryos cultured for 48 hours in 10⁻⁶ M Compound VIII.

FIG. 1D. Transverse histological section through the primary left and right lung buds of three E8.0 embryos cultured for 48 hours in 10⁻⁶ M Compound VIII.

FIG. 1E. Transverse histological section through the primary left and right lung buds of three E8.0 embryos cultured for 48 hours in 10⁻⁶M Compound VIII and 10⁻⁷M RA.

FIG. 1F. Transverse histological section through the primary left and right lung buds of three E8.0 embryos cultured for 48 hours in vehicle (ethanol) alone.

FIG. 1G. Transverse histological section through the primary left and right lung buds of three E8.0 embryos cultured for 48 hours in 10⁻⁶M Compound VIII.

FIGS. 1F, 1G, and 1H represent high power views of FIGS. 1A, 1C, and 1E, respectively. a=dorsal aortas; at=primitive atrium; h=heart outflow tract; I=left lung bud; n=neural tube; r=right lung bud; the arrow heads indicate oesophagotracheal folds. Scale bars=65 μm.

FIG. 2. Effects of RAR agonists and antagonists on E11.75 wild type lungs after four days in culture.

FIG. 2A. Average terminal bud number (ATBN) versus treatment with Compound VIII and RA. Values are calculated from the average percentages with respect to control ATBN from 21 individual experiments each containing ten explants per treatment group. Treatment with 10⁻⁶ M and 10⁻⁷ M RA always resulted in a significant decrease in ATBN, an effect negated by the concomitant presence of 10⁻⁶M Compound VIII.

FIG. 2B. ATBN versus treatment with Compound VIII. Values are calculated from the average percentages with respect to control ATBN from 21 individual experiments each containing ten explants per treatment group. Compound VIII treatment alone at either 10⁻⁶ M or 2×10⁻⁶ M resulted in significant increases in ATBN.

FIG. 2C. Increased explant size and terminal bud numbers of explants treated with 10⁻⁶ M Compound VIII are reduced by the concomitant addition of 10⁻⁶ M RA. * p<0.05 relative to controls. Error bars represent ^{+/-} standard deviation. Scale bar=200 μm.

FIG. 3. RARβ expression during branching morphogenesis.

FIG. 3A. Whole-mount ISH for detection of RARβ transcripts following 24 hours of exposure of E11.75 lung explants to either retinoid vehicle alone, 10⁻⁶ M RA or 10⁻⁶ M Compound VIII.

FIG. 3B. RNAse protection analyses of RARβ 1/3/4, RARβ2 and vimentin transcripts in lungs explanted at E11.75 and cultured for four days in the presence of retinoids or ethanol; the explants were analyzed four hours after the last addition of the retinoids and/or ethanol to the culture media (2 μg RNA per track; exposure times, four to 24 hours).

FIG. 3C. Appearance of representative E12.5 RARβ, heterozygote (+/−) and null (−/−) mutant lungs from RARβ^{+/-}/RARβ^{+/-} crosses, cultured for 24 hours in the absence of retinoids and then 48 hours in the presence of either vehicle alone or 10⁻⁶ M RA. The accompanying histogram depicts ATBN versus treatment at 72 hours of culture; each group consists of seven explants and is representative of two individual experiments. b1=primary bronchi; b2=secondary bronchi; db=distal bud; m=mesenchyme; * p<0.01 relative to control. Error bars represent +/− standard deviation. Scale bars=100 μm.

FIG. 4. RARγ1 transcript localization and effect of Compound VIII treatment on RARγ^{-/-} lungs.

FIG. 4A. Light field photomicrograph of longitudinal section from E13.5 lung demonstrates that RARγ1 transcripts preferentially localize to the distal budding epithelium (db) and tracheal mesenchyme (t) whereas the regions of the primary (b1) and secondary (b2) bronchi only show a weak ISH signal.

FIG. 4B. Dark field photomicrograph of longitudinal section from E13.5 lung demonstrates that RARγ1 transcripts preferentially localize to the distal budding epithelium (db) and tracheal mesenchyme (t) whereas the regions of the primary (b1) and secondary (b2) bronchi only show a weak ISH signal.

FIG. 4C. E12.5 RARγ wild type (+/+), heterozygote (+/−) and null (−/−) mutant lungs from RARγ^{+/-}/RARγ^{+/-}heterozygote crosses were cultured for 24 hours in the absence of retinoids and then for 72 hours in the presence of 10⁻⁶M Compound VIII (seven explants per group, two individual experiments). No significant differences in the stimulation of ATBN were observed between Compound VIII treated groups. * p<0.05 relative to control. Error bars represent +/− standard deviation. Scale bar=100 μm.

FIG. 5. A role for CRBPI during branching morphogenesis.

FIG. 5A. RNAse protection analysis of CRBPI transcripts in E11.75 wild type lungs cultured for four days in the presence of retinoids or ethanol; the explants were analyzed four hours after the last addition of the retinoids, and/or ethanol to the culture media (2 μg RNA per track; exposure times, four to 24 hours). Six experiments per group, 10 lungs per experiment.

FIG. 5B. ATBN in CRBPI null mutant lungs cultured for four days in the presence of either ethanol alone (control), or 10⁻⁶ M or 10⁻⁷ M Compound VIII. Six explants per group, three individual experiments; * p<0.01 relative to control. Error bars represent +/− standard deviation.

### DETAILED DESCRIPTION OF THE INVENTION

As described herein, it has been discovered that retinoic acid signaling is essential at two stages of prenatal lung development. First, an RA signal transduced through RARα/RXRα heterodimers is required for the evagination of the primary lung bud from the primitive foregut. Subsequently, RA signaling through RARβ inhibits lung branching thereby specifying the morphogenetically inactive regions which form conducting airways.

The invention is directed to a method of treating lung branching malformation in a subject in need thereof, the method comprising administering to the subject a pharmaceutically effective amount of a retinoic acid receptor (RAR) resinoid.

In the invention, the malformation can be due to decreased lung branching. Such malformation can cause, for example, lung hypoplasia. In this aspect of the invention, the RAR retinoid to be administered is an antagonist. The antagonist can be an RARα or RARβ antagonist, preferably an RARβ antagonist. In the invention, the subject can be further administered a pharmaceutically effective amount of a cellular retinol binding protein-I (CRBPI) antagonist. The CRBPI antagonist can be, but is not limited to, a CRBPI antibody, a CRBPI antisense oligonucleotide, a RAR antagonist, and dibutyryl cAMP.

Alternatively, the malformation can be due to increased lung branching. Such malformation can cause, for example, hyperplasia of bronchi and/or alveoli. In this aspect of the invention, the RAR retinoid to be administered is an agonist. The agonist can be an RARα, RARβ or RARγ agonist, preferably an RARβagonist. In the invention, the subject can be further administered a pharmaceutically effective amount of a cellular retinol binding protein-I (CRBPI) agonist. The CRBPI agonist can be, but is not limited to, an RAR agonist, RARγ agonist, retinoic acid, retinol, retinal, 13-cis-retinoic acid, 9-cis-retinoic acid, dexamethasone, triiodothyronine, transforming growth factor β (TGFβ), Ch-55, etretinate, and retinoic acid β-glucuronide.

The invention is also directed to a method of increasing alveoli in a subject in need thereof, the method comprising administering to the subject a pharmaceutically effective amount of a retinoic acid receptor (RAR) antagonist. The RAR antagonist can be an RARα or RARβ antagonist, preferably an RARβ antagonist. The subject can be further administered a pharmaceutically effective amount of a cellular retinol binding protein-I (CRBPI) antagonist. The CRBPI antagonist can be, but is not limited to, a CRBPI antibody, a CRBPI antisense oligonucleotide, a RAR antagonist, and dibutyryl cAMP. The subject can suffer from a disease such as, but not limited to, chronic obstructive pulmonary disease, emphysema, chronic bronchitis, interstitial fibrosis, pulmonary tuberculosis, or sarcoidosis.

The invention is further directed to a method of inducing primary lung bud formation in a subject, the method comprising administering a pharmaceutically effective amount of a retinoic acid receptor agonist (RARα, RARβ, or RARγ agonist).

In the above embodiments, in addition to administering an RAR antagonist or agonist, an RXR antagonist or agonist, respectively, can also be administered to the subject for treatment. It has been shown that an RXR agonist has a synergistic effect on transcriptional activation by an RAR agonist (Dilworth, F. J. et al., Proc. Natl. Acad. Sci. USA 96:1995-2000 (1999)).

The invention is also directed to a method of identifying an agent capable of inducing primary lung bud formation, the method comprising: (a) administering an agent to an embryo; and (b) determining primary lung bud formation of said embryo. In the invention, (b) can be further compared with an embryo untreated with said agent.

Each of the terms and elements of the invention as described in the above embodiments is detailed as follows.

Lung Development and Malformations

Lung development includes: (i) evagination of the primitive lung and tracheal buds from the embryonic foregut, (ii) branching morphogenesis, which essentially takes place during the pseudoglandular stage and establishes the primitive conducting airways and presumptive terminal sacs, and (iii) alveologenesis, characterized by septation of the terminal sacs to form definitive alveoli (Hogan, B. L. and Yingling, J. M., Curr. Opin. Genet. Dev. 8:481-486 (1998) and references therein).

Lung bud formation can be divided into three individual stages:

i) The first morphologically distinguishable event during lung development is the formation of the laryngotracheal groove in the ventral wall of the caudal end of the primitive pharynx at approximately embryonic day 9 (E9) in the mouse. E0.5 represents the morning of appearance of the vaginal plug. The primary buds are the two lung primordia, the presumptive left lung and presumptive right lung, which evert from the distal extremity of the laryngotracheal tube at approximately E9.5 and invade the surrounding mesoderm.

ii) Distal and lateral bud bifurcations form from subsequent primary bud ramifications during a process known as branching morphogenesis. This process occurs during the pseudoglandular stage. These buds define branch points in the developing pulmonary tree and hence determine the general form, structure or shape of each lung and its lobe(s).

iii) Terminal buds are the terminal sacs that develop into alveoli through a process of septation.

A table listing the timing of appearance of principle features in mouse, rat and human embryos is provided in Kaufman, M. H., “The Atlas of Mouse Development,” London Academic Press Limited (San Diego); 1992; p7:

a) Primary bud formation in the mouse=E9-9.5, equivalent to E10.5 in the rat and E24-25 in the human; and b) The pseudoglandular stage begins in the mouse at E9.5, in the rat at E10.5-11 and in the human at E26.

Nelson, O. E., “Comparative Anatomy of the Vertebrates,” The Blakiston Company Inc. (New York 1953), pp. 634-652, provides the following:

a) In the chick, the appearance of the laryngotracheal groove occurs at 52/53 h after incubation, primary bud formation occurs between 53 and 96 hours. Lung bud outgrowth, or bronchi extension occurs at E4. Air sac development occurs between E6 and E12.

b) Laryngotracheal groove formation occurs at week 4 in humans.

According to Patten, B. M., “Embryology of the Pig,” McGraw-Hill Book Company, 3rd Edition (New York 1948), p.188, a4-5 mmpig fetus=embryonic week 4 in human (i.e., approximate time of formation of the primary lung buds) and a 7.5 mm pig fetus=pseudoglandular stage of lung development. According to Rugh, R., “Vertebrate Embryology,” Harcourt, Brace and World Inc. (New York 1964), p. 320, a 6 mm pig (E8)=E4 in the chick and E26 in the human.

Ten Have-Opbroek, A. A., Am. J. Anat. 162:201-19 (1981) provides that the human pseudoglandular stage occurs at 3-16 weeks and the human alveolar stage occurs 6-8 weeks before birth until 8 years in post natal life. Ten Have-Opbroek, A. A., Exp. Lung Res. 17:111-130 (1991), provides that the pseudoglandular stage in the mouse=E9.5-16.5, the canalicular stage in the mouse=E16.6-17.4, the terminal sac stage in the mouse=E17.4*5 days post partum, and the alveolar stage in the mouse=5 to 30 days post partum.

According to Wilson, J. G. et al., Am. J. Anat. 92:199-201 (1953), laryngotracheal groove formation in the rat=E12, primary lung bud formation in the rat=E13, and pseudoglandular stage in the rat=E14 to approximately E16.

As used herein, by primary lung bud formation is intended the first step in lung development wherein the primitive lung and tracheal buds evert from the embryonic foregut. Primary lung bud formation can be determined by methods known in the art, e.g., analyzing serial transverse histological sections of the embryo or embryos following administration of the candidate agent to detect primary lung bud outgrowth. In one aspect of the invention, primary lung bud formation in the sample (e.g., embryo) can be compared to an untreated sample. As used herein, by untreated is intended to refer to a sample which has not been administered a candidate agent.

As used herein, lung branching or lung branching morphogenesis is the generation, formation or development of lung branching to establish the primitive conducting airways and presumptive terminal sacs. This process occurs during the pseudoglandular stage of lung development and involves the formation of distal and lateral buds and branches but does not appear to include primary bud formation. Lung branching can be determined by, for example, counting every terminal bud in each explant following administration of the candidate agent and calculating the average terminal bud number (ATBN).

As used herein, by lung branching malformation is intended a defect in the formation and/or complete absence of lung branches. By lung hypoplasia is intended reduced or impaired lung growth and development, usually associated with a decrease in the number of cells. By lung hyperplasia is intended over-growth and development of lung tissue, usually associated with an increase in the number of cells.

By “increasing alveoli” is intended to include, but is not limited to, generation of alveoli by a process of alveolar growth or re-growth (re-alveolarization) and/or generation or re-generation of alveolar walls (re-septation), including reversal of alveolar destruction. By alveolar destruction is intended dilatation of the terminal air spaces of the lung, distal to the terminal bronchioles, through destruction of their walls. This alveolar destruction is a feature of a number of disease states including, but not limited to, chronic obstructive pulmonary disease (COPD) (e.g., emphysema), sarcoidosis, diffuse interstitial fibrosis, bronchopulmonary dysplasia, pulmonary tuberculosis, and other granulomatous diseases. COPD is a disease of the lungs characterized by the presence of chronic airflow obstruction due to chronic bronchitis and/or emphysema and/or small airways disease. The airflow obstruction is generally progressive, may be accompanied by airway hyperreactivity, and may be partially reversible. The distinctions among the definitions of airway obstruction, chronic bronchitis, chronic obstructive bronchitis, pulmonary emphysema, chronic obstructive emphysema, chronic asthmatic bronchitis, and chronic obstructive pulmonary disease areprovided in “The Merck Manual,” 16th Supp. Ed., pp. 658-659, Merck Research Laboratories, Rahway, N.J., 1992.

The term airway obstruction refers to an increased resistance to airflow exhibited by characteristic spirometric findings. The term chronic bronchitis refers to the condition associated with prolonged exposure to nonspecific bronchial irritants and is accompanied by mucus hypersecretion and structural changes in the bronchi. The term chronic obstructive bronchitis refers to the disease condition frequently associated with the symptoms of chronic bronchitis in which disease of the small airways has progressed to the point that there is clinically significant airway obstruction. The term pulmonary emphysema refers to enlargement of the airspaces distal to the terminal nonrespiratory bronchioles, accompanied by destructive changes of the alveolar walls. The term chronic obstructive emphysema refers to the condition when there has been sufficient loss of lung recoil to allow marked airway collapse upon expiration, leading to the physiologic pattern of airway obstruction. The term chronic asthmatic bronchitis refers to an underlying asthmatic condition in patients in whom asthma has become so persistent that clinically significant chronic airflow obstruction is present despite antiasthmatic therapy.

Details of primary lung bud formation and lung branching are set forth in Warburton, D. et al., Mech. Dev. 92:55-81 (2000), and Metzger, R. J. and Krasnow, M. A., Science 284:1635-9 (1999).

By “subject” is intended a postnatal animal, mammal or nonmammal, human or nonhuman (e.g., rat, mouse, pig, sheep, chick) or an embryo or fetus (prenatal) of such. By postnatal is intended life after birth of the subject, including adult life. By prenatal is intended life before birth. The subject can suffer from a disease state, as described above. By “suffer” is intended experiencing or enduring a disease, illness, condition, or the symptoms thereof, or having predisposition to a disease, illness, condition, or the symptoms thereof. In the invention, appropriate agent(s) (RAR antagonist or agonist, RXR antagonist or agonist, or CRBPI agonist or antagonist) can be administered prenatally or postnatally, depending on the need of the subject and result intended in the subject, based on the teachings herein and knowledge in the art.

Based on the lung developmental stages discussed herein and based on the knowledge in the art, the invention can be practiced at appropriate stages of development and postnatal life. For example, a subject can be treated as follows:

i) treat with an RAR agonist between approximately E8.0 and E9.5 in mice, E9.5 and E11.5 in rats, E4 and ES in pigs, and E21 and E25 in humans and 48 and 51 hours post incubation in the chick, to induce or augment primary lung bud formation,

ii) treat with an RAR antagonist at approximately E11.75 to E16.5 in mice, E13.5 to E16 in rats, E10 to E14 in pigs, and embryonic week 3 to embryonic week 16 in human to induce or augment lung branching morphogenesis,

iii) treat with an RAR agonist at approximately E11.75 to E16.5 in mice, E13.5 to E16 in rats, E10 to E14 in pigs, and embryonic week 5 to embryonic week 16 in human to inhibit lung branching morphogenesis,

In the invention, the appropriate agent(s) as specified herein can be administered postnatally for treating lung malformation or for generating alveoli. An RAR retinoid (antagonist) can also be administered to a human 6-8 weeks before birth or thereafter for alveolar generation.

CRBPI Agonists and Antagonists

Cellular retinol binding protein-I (CRBPI) is a cytoplasmic binding protein. It has been discovered that absence of CRBPI synergizes RAR antagonist induced augmentation of lung branching at the pseudoglandular stage of lung development.

By “CRBPI agonist” is intended a compound or molecule which increases CRBPI function by mediating the binding of retinol to CRBPI, thereby increasing its function, or increases CRBPI level, such as its mRNA level. CRBPI agonists include, but are not limited to, a RAR agonist, RARγ agonist (Chiba, H. et al., Mol. Cell Biol. 17:3013-20 (1997)), retinoic acid, retinol, retinal, 13-cis-retinoic acid, 9-cis-retinoic acid, dexamethasone, triiodothyronine (Okuna, M. et al., J. Lipid Res. 36:137-47 (1995)), transforming growth factor β (Xu, G. et al., Amer. J. Pathol. 151:1741-9 (1997)), Ch-55 (Kooistra, T. et al., Euro. J. Biochem. 232:425 (1995)), etretinate, and retinoic acid β-glucuronide (Hamish, D. C. et al., Teratology 46:136-46 (1992)).

By “CRBPI antagonist” is intended a compound or molecule which decreases CRBPI function by inhibiting or interfering with the binding of retinol to CRBPI, thereby decreasing its function, or decreases CRBPI level, such as its mRNA level. CRBPI antagonists include, but are not limited to, a CRBPI antibody, a CRBPI antisense oligonucleotide, a RAR antagonist, and dibutyryl cAMP (Oyen, O. et al., Mol. Endocrinol. 2:1070-1076 (1988)).

The antibodies used in the invention can be, but are not limited to, chimeric, humanized, and human and nonhuman monoclonal and polyclonal antibodies. The antibodies may be prepared by any suitable method known in the art. For example, a polypeptide of the present invention or an antigenic fragment thereof can be administered to an animal in order to induce the production of sera containing polyclonal antibodies. Monoclonal antibodies can be prepared using a wide of techniques known in the art including the use of hybridoma and recombinant technology. See, e.g., Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS 563-681 (Elsevier, N.Y., 1981) (said references incorporated by reference in their entireties).

Antisense technology can be used to control expression of the CRBPI gene through antisense DNA or RNA or through triple-helix formation. Antisense techniques are discussed, for example, in Okano, J. Neurochem. 56:560 (1991); Gewirtz, A. M. et al., Blood 92:712-36 (1998); Roush, W., Science 276:1192-3 (1997); Strauss, E., Science 286:886 (1999); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1360 (1991). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of CRBPI. The antisense RNA oligonucleotide hybridizes to the mRNA in vivo and blocks translation of the m mRNA molecule into the CRBPI polypeptide. The oligonucleotides described above can also be delivered to cells such that the antisense RNA or DNA may be expressed in vivo to inhibit production of CRBPI.

This oligonucleotide may be delivered to cells in a number of forms, including as antisense RNA or incorporated into an expression vector. If incorporated into an expression vector, the oligonucleotide is generally orientated in a manner that an RNA molecule is produced upon in vivo expression which is complementary to that of the CRBPI mRNA sequence. The expressed antisense RNA molecule will hybridize to CRBPI mRNA and block translation in vivo.

RAR and RXR Antagonists and Agonists

A “retinoid” is a compound which binds to one or more of the retinoid receptors (RARα, RARβ, RARγ, RXRα, RXRβ and RXRγ). Compounds are either “RAR retinoids” or “RXR retinoids” depending on their binding characteristics (RAR retinoids bind to one or more RARs; RXR retinoids bind to one or more RXRs (also referred to as “rexinoids”)). Retinoids which cause transactivation via their receptors are examples of “agonists,” while retinoids which do not cause transactivation, but instead block the transactivation caused by other agonists, are examples of “antagonists.” RXR and RAR antagonists and agonists to be used in the methods of the present invention can be, but are not limited to, peptides, carbohydrates, steroids and vitamin derivatives, which can each be natural or synthetic (prepared, for example, using methods of synthetic organic and inorganic chemistry that are well-known in the art).

In the invention, the retinoids can be nonspecific, nonselective, specific or selective. By retinoids that are “specific” for a retinoid receptor are intended compounds that only bind to a particular retinoid receptor (RARα, RARβ, RARγ, RXRα, RXRβ, or RXRγ). By retinoids that are “selective” for a retinoid receptor are intended compounds that preferably bind to a particular retinoid receptor over others by a magnitude of approximately five-fold or greater than to other retinoid receptors, preferably eight-fold or greater, more preferably, ten-fold or greater. In the invention, the retinoids can be selective for one or more retinoid receptors. In the invention, unless specified, “RARβ antagonist,” for example, is intended an antagonist that blocks transactivation by agonists to RARβ and can or cannot block transactivation by agonists to other retinoid receptors.

Standard retinoids known in the art as RAR agonists include the following:

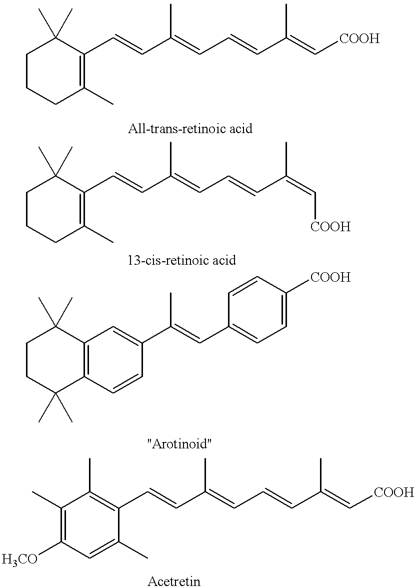

RARα,β-selective agonists include, but are not limited to,

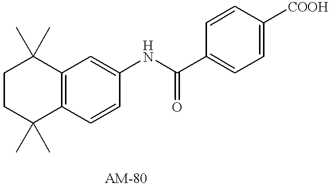

(see, Takeuchi, M., et al., Brit. J. Haematol. 97:137-140 (1997)).

RARβ,γ-selective agonists include, but are not limited to,

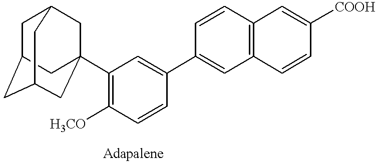

(see, Shroot, B. and Michel, S., J. Amer. Acad. Dermatol. 36: S96-S103 (1997)).

RARγ agonists include, but are not limited to,

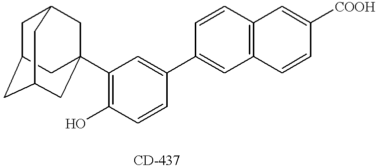

(see, Schadendorf, D., et al., Intl. J. Oncol. 5:1325-1331 (1994)); and

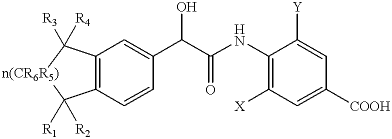

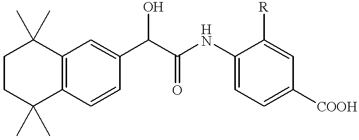

(see, Swann, R. T., et al., EP 747,347).

RAR agonists include, but are not limited to,

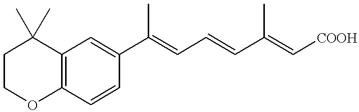

(see, Benbrook, D. M., et al., J. Med. Chem. 40:3567-3583 (1997));

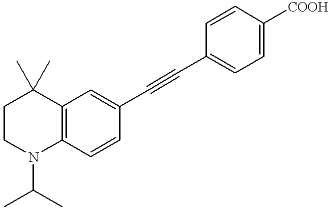

(see, Beard, R. L., et al, Bioorg. Med. Chem. Lett. 7:2372-2378 (1997)); and

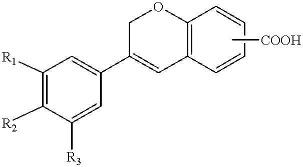

(see, Diaz, P., et al., Bioorg. Med. Chem. Lett. 7:2289-2294 (1997)).

RARα antagonists include, but are not limited to,

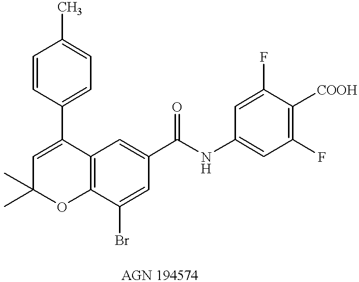

(see, Teng, M., et al., J. Med. Chem. 40:2445-2451 (1997)).

RARα,β antagonists include, but are not limited to,

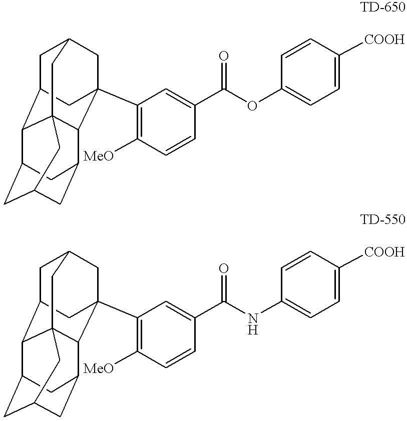

(see, Kaneko, S., et al., Med. Chem. Res. 1:220-225 (1991)).

RAR antagonists include, but are not limited to,

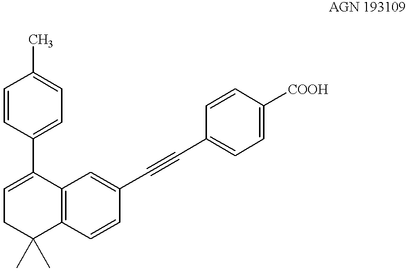

(see, Agarwal, C., et al., J. Biol. Chem. 271:12209-12212 (1996); Johnson, A. T., et al., J. Med. Chem. 38:4764 (1995); Klein, E., et al., WO 97/09,297);

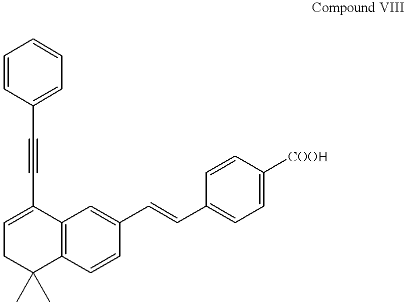

(see Tramposch, K. M. et al., WO 98/46228).

Further, RARα specific or selective agonists and antagonists can contain an amide group. RARγ specific or selective agonists can contain a hydroxyl group or a carbonyl group such as a flavone structure. RARβ specific or selective agonists can be characterized by the absence of a hydroxy and amide groups. Moreover, it has been determined that RARβ specific agonists can be characterized by a dihydronaphthalene nucleus bearing a 2-thienyl group at C8 (see, U.S. Pat. No. 5,559,248; Johnson, A. T., et al., J. Med. Chem. 39:5029-5030 (1996)).

RXR antagonists include, but are not limited to,

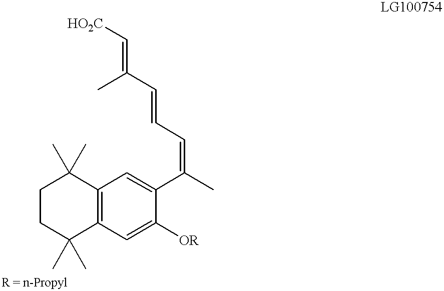

(see, Canan Koch, S. S., et al., J. Med. Chem. 39:3229-3234 (1996); and

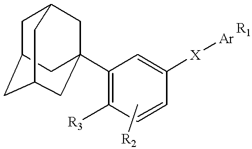

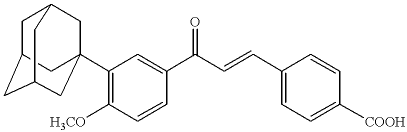

(see, Bemardon, J. M. and B. Charpentier, EP 776,881).

General RXR agonists include, but are not limited to,

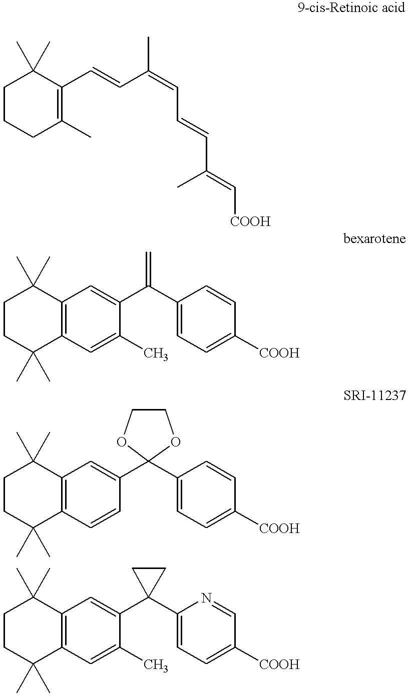

Additional RXR agonists include, but are not limited to,

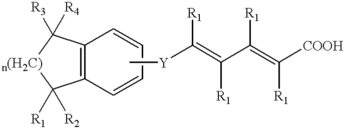

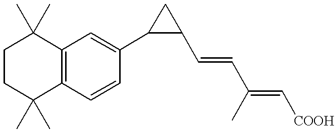

(see, Vuligonda, V. And R. A. Chandraratna, U.S. Pat. No. 5,675,033);

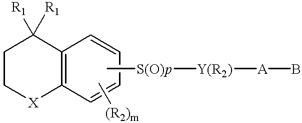

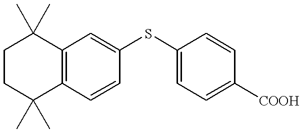

(see, Beard, R. L., et al., WO 97/16,422);

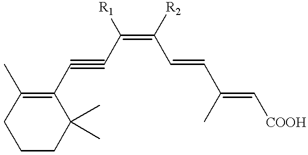

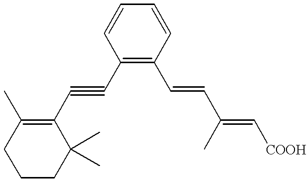

(see, Klaus, M., et al., EP 728,742);

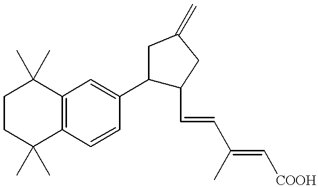

(see, Farmer, L. J., et al., Bioorg. Med. Chem. Lett. 7:2393-2398 (1997)); and

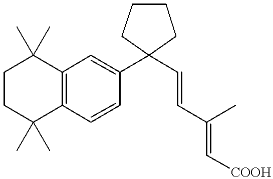

(see, Farmer, L. J., et al., Bioorg. Med. Chem. Lett. 7:2747-2752 (1997)).

RAR or RXR agonists include, but are not limited to,

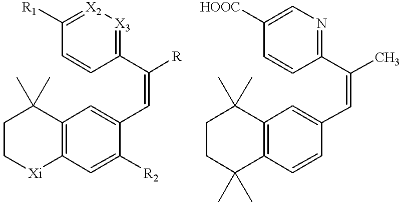

(Leblond, B., WO 97/26,237).

Other RXR agonists, with a variety of structures, are disclosed in Boehm, M. F., et al., J. Med. Chem. 38:3146-3155 (1995). Further, a number of retinoids of diverse structure types which are triple RAR agonists, selective RARα agonists, selective RARβ agonists, selective RARγ agonists, selective RARβ,γ agonists, selective RXR agonists and RXR/RAR pan-agonists are described in Sun, S. Y., et al, Cancer Res. 57:4931-4939 (1997). The structure and preparation of RXR agonist bexarotene are described in Boehm et al., J. Med. Chem. 37:2930-2941(1994). Other RXR agonists are also described in, for example, Lehmann et al, Science 258:1944-1946 (1992).

Other candidate RAR and/or RXR agonists include, but are not limited to,

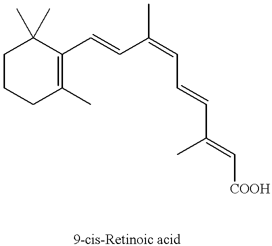

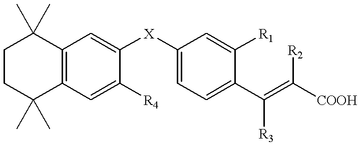

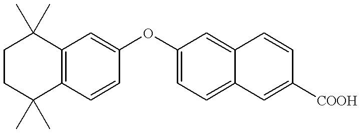

(see, Bemardon, J. M., EP 722,928);

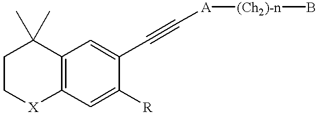

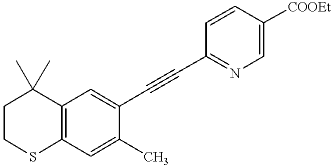

(see, Chandraratna, R., WO 96/11,686; and Drugs of the Future 22:249-255 (1997));

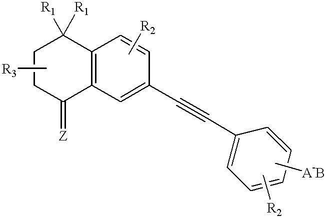

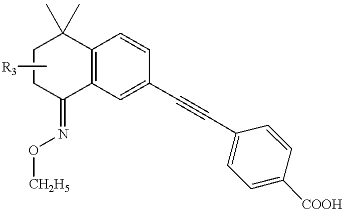

(see, Vuligonda, S., et al., U.S. Pat. No. 5,599,967);

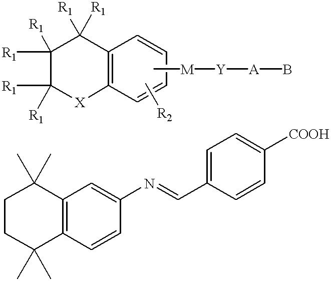

(see, Chandraratna, R. A. and M. Teng, WO 96/06,070);

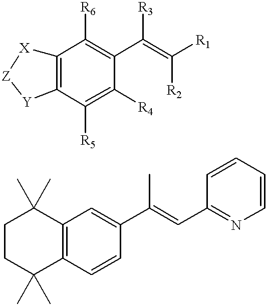

(see, Klaus, M. and E. Weis, EP 253,302);

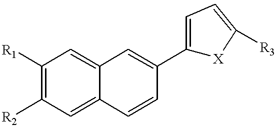

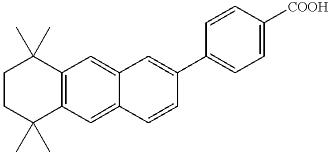

(see, Shroot, B. V., et al., EP 210,929); and

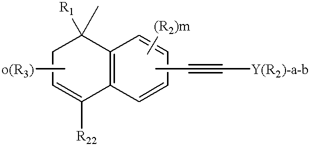

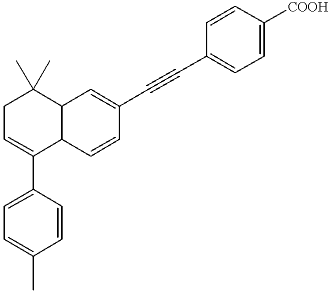

(see, Johnson, A. T., et al., U.S. Pat. No. 5,648,514).

Thus, preferred RXR agonists that can be used in the invention include, but are not limited to, 9-cis retinoic acid, 4-[1-[5,6-Dihydro-3,5,5-trimethyl-8-(1-methylethyl)-2-naphthalenyl]ethenyl]benzoic acid (structure and synthesis provided in U.S. Appl. No. 60/127,976, filed Apr. 6, 1999, titled “Selective Retinoic Acid Analogs” (Atty. Docket: SD128*); and U.S. Appl. No. 60/130,649, filed Apr. 22, 1999, titled “Selective Retinoic Acid Analogs” (Atty. Docket: SD128a*)), SR11237 (structure and synthesis provided in U.S. Pat. No. 5,552,271), and bexarotene. RARα agonists that can be used in the invention include, but are not limited to, all-trans retinoic acid, 4-[[(2,3-Dihydro-1,1,3,3 -tetramethyl-2-oxo-1H-inden-5-yl)carbonyl]amino]benzoic acid (structure and synthesis provided in WO 98/47861), AM-80 and AM-580.

A preferred pan-RAR antagonist is Compound VIII (WO 98/46228):

| Compound | Structure | CAS Name |
|---|---|---|
| VIII | 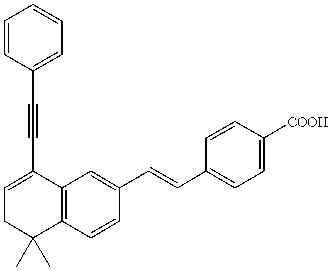 | 4-[(E)-2-[5,6-dihydro- 5,5-dimethyl-8- phenylethynyl)-2- naphthalenyl]ethenyl]benzoic acid |

Other RXR antagonists, RXR agonists, RAR antagonists and RAR agonists suitable for use in the present invention can be prepared by the below-cited methods and others routine to those of ordinary skill in the art.

Screening Methods

A number of methods for screening candidate and identifying RAR and RXR agonists and antagonists are well-known in the art, and will allow one of ordinary skill to determine if a compound is useful in the present invention.

The agent can be selected and screened at random, or can be rationally selected or rationally designed using protein modeling techniques.

For random screening, agents such as, but not limited to, peptides, carbohydrates, steroids or vitamin derivatives (e.g., derivatives of retinoic acid) are selected at random and are assayed, using direct or indirect methods that are routine in the art, for their ability to bind to a retinoid receptor or a functional retinoid receptor heterodimer that is present in mice or cell lines described in the present invention. Alternatively, agents can be assayed for retinoic acid agonist or antagonist activity.

Agents can be rationally selected. As used herein, an agent is said to be “rationally selected” when the agent is chosen based on the physical structure of a known ligand of a retinoid receptor or a functional heterodimeric retinoid receptor. For example, assaying compounds possessing a retinol-like structure would be considered a rational selection since retinol-like compounds are known to bind to a variety of retinoid receptor heterodimers.

Since highly purified RAR and RXR proteins are now available, X-ray crystallography and NMR-imaging techniques can be used to identify the structure of the ligand binding site present on these proteins and, by extension, that which is specifically present on the retinoid receptors. Utilizing such information, computer modeling systems are now available that allows one to “rationally design” an agent capable of binding to such a defined structure (Hodgson, Biotechnology 8:1245-1247 (1990)), Hodgson, Biotechnology 9:609-613 (1991)).

As used herein, an agent is said to be “rationally designed” if it is selected based on a computer model of the ligand binding site of one or more retinoid receptor(s).

For example, in Chen, J. -Y. et al, EMBO J. 14:1187-1197 (1995), three “reporter” cell lines have been used to characterize a number of RARα-, RARβ-, or RARγ-specific dissociating synthetic retinoids that selectively induce the AF-2 activation function present in the LBD of RARβ (βAF-2). These cell lines stably express chimeric proteins containing the DNA binding domain of the yeast transactivator GAL4 fused to the EF regions (which contain the LBD and AF-2 activation function) of RARα (GAL-RARα), RARβ (GAL-RARβ) or RARγ (GAL-RARγ), and a luciferase reporter gene driven by a pentamer of the GAL4 recognition sequence (“17m”) in front of the β-globin promoter ((17m)5-GAL-Luc). In these cell lines, the RAR ligands thus induce luciferase activity that can be measured in the intact cells using a single-photon-counting camera. This reporter system is insensitive to endogenous receptors which cannot recognize the GAL4 binding site. Using analogous screening assays, these synthetic retinoids, like RA, have been reported to inhibit the anchorage-independent growth of oncogene-transformed 3T3 cells, while the promoter of the human interleukin-6 (IL-6) gene, whose product is involved in the regulation of hematopoiesis, immune responses and inflammation (Kishimoto, T., et al., Science 258:593-597 (1992)) has been shown to be induced by RA but not by the synthetic dissociating retinoids which repressed its activity.

In a similar manner, RXR agonists have been identified using cell lines that express a RXR receptor linked to a TREpal-tk reporter gene which is activated by both RAR/RXR heterodimers and RXR homodimers (Lehmann, J. M., et al., Science 258:1944-1946 (1992)). Thus, reporter cell lines that are easily constructed, by methods routine to one of ordinary skill, can be used to distinguish not only the specific RAR or RXR types to which a candidate ligand will bind, but also whether that binding induces an activating (i.e., agonistic) or repressive (i.e., antagonistic) effect. Although the above-referenced reporter cell lines comprised the luciferase or thymidine kinase genes as reporters, other reporters such as Neo, CAT, β-galactosidase or Green Fluorescent Protein are well known in the art and can be used in a similar fashion to carry out the present invention. For example, references disclosing reporter plasmids containing a reporter gene and expression vectors encoding a LBD of a nuclear receptor include Meyer et al., Cell 57:433-442 (1989); Meyer et al., EMBO J. 9:3923-3932 (1990); Tasset et al., Cell 62:1177-1187 (1990); Gronemeyer, H., and Laudet, V., Protein Profile 2:1173-1308 (1995); Webster et al., Cell 54:199-207 (1988); Strahle et al., EMBO J. 7:3389-3395 (1988); Seipel et al., EMBO J. 11:4961-4968 (1992); and Nagpal, S., et al., EMBO J. 12:2349-2360 (1993).

Other routine assays have been used to screen compounds for their agonistic or antagonistic properties on functions of other nuclear receptors, such as steroid receptors. For example, a transient expression/gel retardation system has been used to study the effects of the synthetic steroids RU486 and R5020 on glucocorticoid and progesterone receptor function (Meyer, M. -E., et al., EMBO J. 9:3923-3932 (1990)). Similar assays have been used to show that tamoxifen competitively inhibits estradiol-induced ERAP160 binding to the estrogen receptor, suggesting a mechanism for its growth-inhibitory effects in breast cancer (Halachimi, S., et al., Science 264:1455-1458(1994)). Since the RAR and RXR receptors are apparently structurally similar to other nuclear receptors such as the steroid receptors (as reviewed in Chambon, P., FASEB J. 10:940-954 (1996)), routine assays of this type can be useful in assessing compounds for their agonistic or antagonistic activities on RAR and/or RXR receptors.

As an alternative routine method, the effect of a candidate agonist or antagonist on the binding of the ligand-dependent AF-2 modulator TIF1 to a RAR or RXR LBD can be studied using glutathione-S-transferase (GST) interaction assays by tagging the LBDs with GST as described in detail in Le Douarin et al., EMBO J. 14:2020-2033 (1995).

In another screening assay, transgenic animals, e.g., mice, and cell lines, that are altered in their expression of one or more of RAR and RXR receptors can be made as described previously (Krezel, W., et al., Proc. Natl. Acad. Sci. USA 93:9010-9014 (1996)) and can be used to identify agonists and antagonists of specific members of the RAR/RXR class of receptors using methods described previously (WO 94/26100). In such an assay, the agent which is to be tested will be incubated with one or more of the transgenic cell lines or mice or tissues derived therefrom. The level of binding of the agent is then determined, or the effect the agent has on biological effect or gene expression is monitored, by techniques that are routine to those of ordinary skill. As used herein, the term “incubate” is defined as contacting the compound or agent under investigation with the appropriate cell or tissue, or administering the agent or compound to the appropriate animal, e.g., transgenic mouse, via any one of the well-known routes of administration including enteral, intravenous, subcutaneous, and intramuscular.

Other assays can also be used to determine the agonistic or antagonistic effects of RAR and RXR ligands. For example, certain agonistic retinoids will induce the association of endogenous PML/PML-RARα fusion protein with nuclear bodies in cells from APL patients (Dyck, J. A., et al., Cell 76:333-343 (1994); Weis, K., et al., Cell 76:345-356 (1994); Koken, M. H. M., et al., EMBO J. 13:1073-1083 (1994)) or in related established cell lines such as NB4 (Lanotte, M., et al., Blood 77:1080-1086 (1991)). These effects of RAR or RXR agonists or antagonists can be determined, for example, by various immunological techniques such as immunofluorescent or immunoelectron microscopy, using antibodies specific for PML, RAR and/or PML-RARα fusion proteins. RAR or RXR agonists or antagonists can also be identified by their abilities to induce the in vitro differentiation (maturation) of certain established cell lines such as HL-60 myeloblastic leukemia cells (Nagy, L., et al., Mol. Cell. Biol. 15:3540-3551 (1995)), NB4 promyelocytic cells (Lanotte, M., et al., Blood 77:1080-1086 (1991), P19 or F9 embryonic carcinoma cells (Roy, B., et al., Mol. Cell. Biol. 15:6481-6487 (1995); Horn, V., et al., FASEB J. 10:1071-1077 (1996)), or ras-transformed 3T3 cells (Chen et al., EMBO J. 14:1187-1197 (1995)). Ligand-induced differentiation in these and other cell lines can be determined by assaying ligand-treated or -untreated cells for the expression of a variety of well-known markers of differentiation as generally described in the above references.

Similarly, the candidate antagonists or agonists can be screened by measuring their abilities to induce apoptosis (programmed cell death) in, for example, HL-60 cells (Nagy, L., et al., Mol. Cell. Biol. 15:3540-3551 (1995)) or P19 cells (Horn, V., et al., FASEB J. 10: 1071-1077 (1996)), or in other primary cells or established cell lines. Apoptosis is typically assessed by measurement of ligand-induced DNA fragmentation, which is accomplished by methods such as gel electrophoresis (appearance of smaller molecular weight bands), microscopy (changes in plasma membrane morphology such as formation of surface protruberances (“blebbing”) or in nuclear morphology such as pycnosis or fragmentation) or expression of the putative apoptosis suppressive protein BCL-2 (decreased in apoptotic cells); for general methods and discussions of these assays as they pertain to RAR and RXR biology, see Nagy, L., et al., Mol Cell. Biol. 15:3540-3551 (1995); Horn, V., et al., FASEB J. 10:1071-1077 (1996)). Other methods for assaying ligand-induced apoptosis in primary cells and established cell lines, such as flow cytometry or particle analysis (appearance of smaller particles with different light scatter and/or DNA content profiles), are well-known in the art (Telford, W. G., et al., J. Immunol. Meth. 172:1-16 (1994); Campana, D., et al., Cytometry 18:68-74 (1994); Sgonc, R. and Wick, G., Int. Arch. Allergy Immunol. 105:327-332 (1994); Fraker, P. J., et al., Meth. Cell Biol. 46:57-76 (1995); Sherwood, S. W., and Schimke, R. T., Meth. Cell Biol. 46:77-97 (1995); Carbonari, M., et al., Cytometry 22:161-167 (1995); Mastrangelo, A. J. and Betenbaugh, M. J., Curr. Opin. Biotechnol. 6:198-202 (1995)).

Screening of agonists or antagonists can be accomplished by an assay known as “in vivo footprinting” (Mueller, P. R., and Wold, B., Science 246:780-786 (1989); Garrity, P. A., and Wold, B. J., Proc. Natl. Acad. Sci. USA 89:1021-1025 (1992)), which has proven useful for analysis of RA-induced transcription of RARβ2 (Dey, A., et al., Mol. Cell. Biol. 14:8191-8201 (1994)).

Other methods for determining the agonistic or antagonistic activities of a candidate ligand which are routine in the art can also be used in carrying out the present invention. In performing such assays, one skilled in the art will be able to determine which RAR or RXR receptor subtype an agent binds to, what specific receptor(s) are utilized by a given compound, and whether the agent is an agonist or antagonist of the given receptor(s). CRBPI agonists and antagonists can similarly be screened.

Formulations and Methods of Administration

The term “subject in need thereof” as used herein is intended a subject in need of a meaningful therapeutic benefit prenatally or postnatally. As used herein, “a pharmaceutically effective amount” with respect to an agent (RAR agonists, RAR antagonists, RXR agonists, RXR antagonists, CRBPI agonists, or CRBPI antagonists) is intended to refer to an amount effective to elicit the intended cellular response that is clinically significant, without excessive levels of side effects.

Pharmaceutical compositions are thus provided comprising one or more of RAR agonist, RAR antagonist, RXR agonist, RXR antagonist, CRBPI agonist, or CRBPI antagonist, and a pharmaceutically acceptable carrier or excipient, which can be administered orally, rectally, parenterally, intrasystemically, intravaginally, intraperitoneally, topically (as by powders, ointments, drops or transdermal patch), bucally, or as an oral or nasal spray. By “pharmaceutically acceptable carrier” is intended, but not limited to, a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

Pharmaceutical compositions of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactant.

The compositions of the present invention can also contain adjuvants such as, but not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form can be brought about by the inclusion of agents which delay absorption such as aluminum monostearate and gelatin.

In some cases, in order to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are mixed with at least one item pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hardfilled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, can be prepared as a dry powder which can be pressurized or non-pressurized. In nonpressurized powder compositions, the active ingredients in finely divided form can be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

Alternatively, the composition can be pressurized and contain a compressed gas, such as nitrogen or a liquefied gas propellant. The liquefied propellant medium and indeed the total composition is preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

Compositions for rectal or vaginal administration are preferably suppositories which can be prepared by mixing the agent(s) with suitable non-irritating excipients or carriers such as cocoa butter, polyethylene glycol or a suppository wax which are solid at room temperature but liquid at body temperature and therefore melt in the rectum or vaginal cavity and release the drugs.

The compositions of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the agent(s), stabilizers, preservatives, excipients, and the like. The preferred lipids are the phospholipids and the phosphatidyl cholines (lecithins), both natural and synthetic. Methods to form liposomes are known in the art (see, for example, Prescott, Ed., Meth. Cell Biol. 14:33 et seq (1976)).

Dosaging

One of ordinary skill will appreciate that effective amounts of agent(s) (RAR agonist, RAR antagonist, RXR agonist, RXR antagonist, CRBPI agonist, or CRBPI antagonist) can be determined empirically and can be employed in pure form or, where such forms exist, in pharmaceutically acceptable salt, ester or prodrug form. Such agents can be administered to a patient in need thereof as pharmaceutical compositions in combination with one or more pharmaceutically acceptable excipients. It will be understood that, when administered to a human patient, the total daily usage of the compounds and compositions of the present invention will be decided by the attending physician within the scope of sound medical judgement. The specific therapeutically effective dose level for any particular patient will depend upon a variety of factors: the type and degree of the cellular response to be achieved; activity of the specific agent(s) employed; the specific composition employed; the age, body weight, general health, sex and diet of the patient; the time of administration, route of administration, and rate of excretion of such agent(s); the duration of the treatment; drugs used in combination or coincidental with the specific agent(s); and like factors well known in the medical arts. For example, it is well within the skill of the art to start doses of such agent(s) at levels lower than those required to achieve the desired therapeutic effect and to gradually increase the dosages until the desired effect is achieved.

For example, satisfactory results are obtained by oral administration of such agent(s) at dosages on the order of from 0.05 to 20 mg/kg/day, preferably, 1.0 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 10 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v. Dosaging of the RAR antagonist can be arranged as described in EP 0 661 259 Al (see also, Wendling, O. et al., Development 127:1553-1562 (2000)).

Dosaging can also be arranged in a patient specific manner to provide a predetermined concentration of such agent(s) in the blood, as determined by techniques accepted and routine in the art (HPLC is preferred). Thus patient dosaging can be adjusted to achieve regular on-going blood levels, as measured by HPLC, on the order of from 50 to 1000 ng/ml, preferably 150 to 500 ng/ml.

It will be readily apparent to one of ordinary skill in the relevant arts that other suitable modifications and adaptations to the methods and applications described herein may be made without departing from the scope of the invention or any embodiment thereof.

The following Example serves only to illustrate the invention, and is not to be construed as in any way limiting the invention.

### EXAMPLE

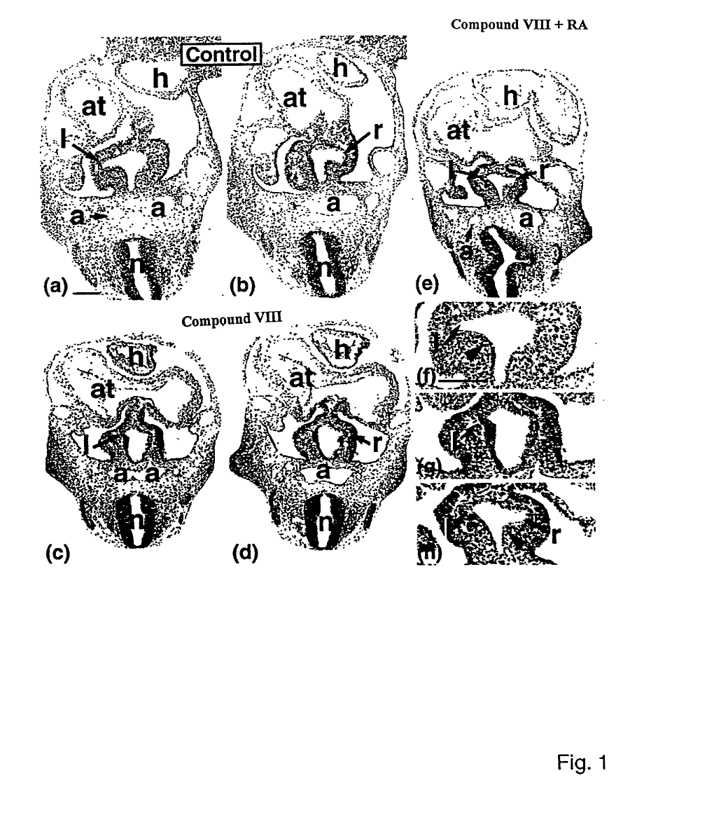

Embryos were collected at E8.0 (i.e. 36 hours prior to the formation of primary lung buds and tracheal diverticulum; Kaufman, M. H., The Atlas of Mouse Development, London Academic Press (1992)), and cultured for 48 hours (i.e. until the equivalent of E9.5 in vivo) either in the presence of the pan-RAR antagonist, Compound VIII (Chazaud, C., et al., Development 126:2589-2596 (1999); Wendling, O., et al., Development 127:1553-62 (2000), or in the presence of the retinoid vehicle (i.e. ethanol alone). Treatment with 10⁻⁶ M Compound VIII, inhibited primary lung bud outgrowth (FIGS. 1A-1D; l and r) and caused a failure of oesophagotracheal fold formation (arrowheads in FIG. 1F compare with FIG. 1G). These defects were partially prevented by the simultaneous addition to the culture medium of 10⁻⁶ M Compound VIII and 10⁻⁷ M RA (FIGS. 1E and 1H; l, r and arrowhead). Therefore, a block in RA signal transduction before and at the onset of lung bud appearance inhibits the formation of the respiratory system from the primitive foregut.

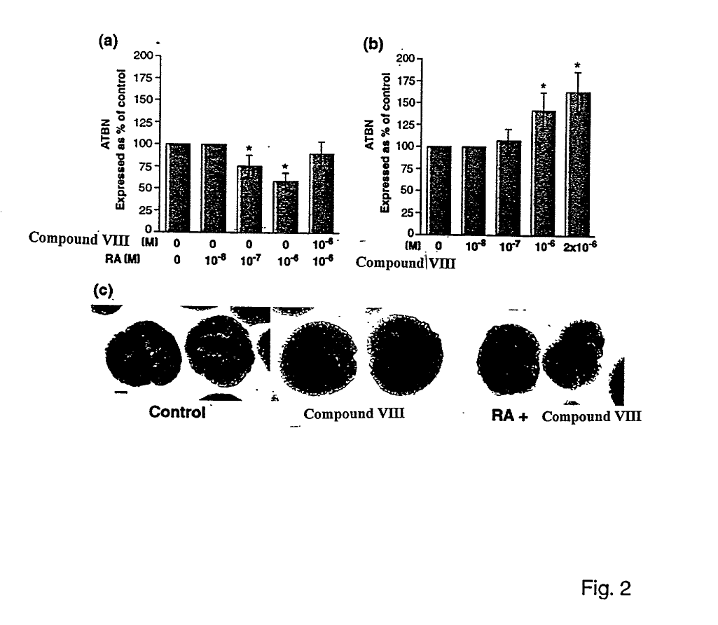

Lung explants from E11.75 or E12.5 embryos were cultured for 4 days, unless otherwise indicated. Control explants were cultured in the presence of the retinoid vehicle alone. RA at final concentrations of 10⁻⁷ M and 10⁻⁶ M significantly decreased average terminal bud number (ATBN) in a dose-dependent fashion, while 10⁻⁸ M was ineffective (FIG. 2A). In contrast, Compound VIII at final concentrations of 10⁻⁶ M and 2×10⁻⁶ M significantly increased ATBN in a dose-dependent manner, while 10⁻⁷ M and 10⁻⁸ M were ineffective (FIGS. 2B and 2C). Retinoid-induced changes in ATBN were prevented when 10⁻⁶ M RA and 10⁻⁶ M Compound VIII were simultaneously added to the culture medium, indicating that the effects of Compound VIII are caused by a specific inhibition of RA-signaling (FIGS. 2A and 2C). Altogether, these data suggest that retinoid signaling decreases branching during the pseudoglandular stage of lung morphogenesis.

During lung development, RARβ is expressed at high levels in the epithelium and mesenchyme of proximal primary and secondary bronchi (Dollé, P., et al., Development 110:1133-1151 (1990); Ghyselinck, N. B., et al., Dev. Biol. 198:303-318 (1998) and see below) which correspond to areas of morphogenetic stability when compared to the morphogenetically active distal buds (Hilfer, S. R., et al., Tissue Cell 17:523-538 (1985); Mollard, R. and Dziadek, M., Am. J. Respir. Cell Mol. Biol. 19:71-82 (1998)).

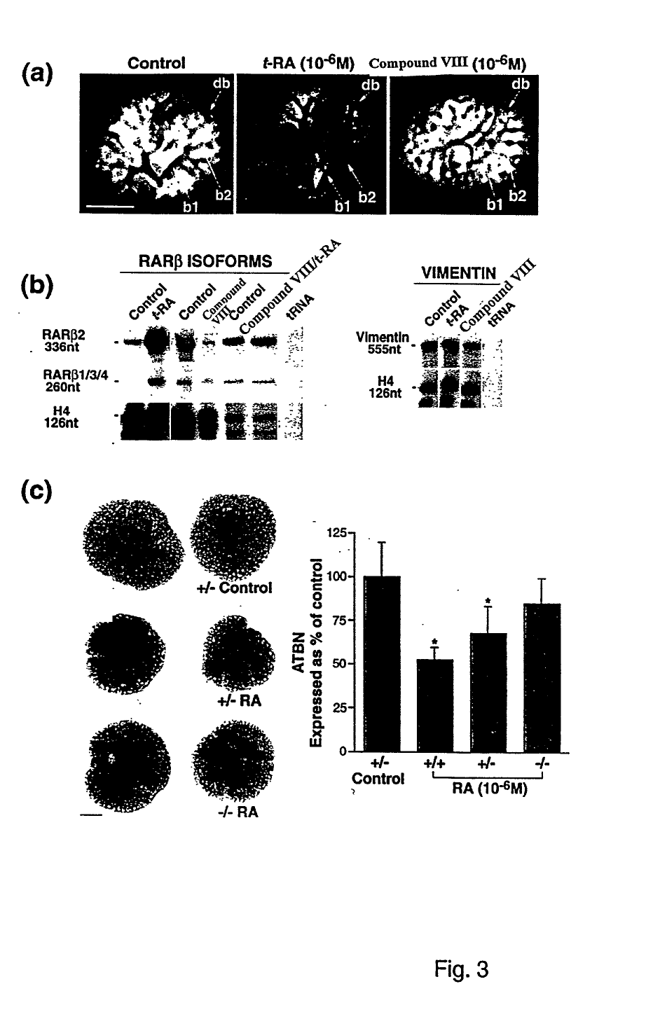

The expression pattern of RARβ transcripts in E11.75 lung explants cultured for 24 hours in the absence of retinoids was similar to that observed in vivo at E13.5 (FIG. 3A). RA treatment at 10⁻⁶M induced the expression of RARβ throughout the pulmonary tree, including the distal buds, whereas treatment with Compound VIII (10⁻⁶M) decreased RARβ expression in secondary bronchi (FIGS. 3A and 3B). Retinoid-induced changes in RARβ expression were prevented when 10⁻⁶ M RA and 10⁻⁶ M Compound VIII were simultaneously added to the culture medium (FIG. 3B), indicating that competition between RA and Compound VIII modified transcription in explanted lungs, as previously observed in cultured cells. It is also noteworthy that only limited changes in the expression of vimentin, a specific mesenchymal marker, occurred following retinoid treatment (FIG. 3B). Therefore, a modification in the epithelial to mesenchymal ratio presumably does not account for the alterations in RARβ transcription levels. Altogether, these results suggest that RARβ could mediate the observed RA-induced inhibition of lung branching. In order to investigate this possibility, we compared the effects of 10⁻⁶ M RA upon ATBN in explants from RARβ null embryos (Ghyselinck, N. B., et al., Int. J. Dev. Biol. 41:425-447 (1997)), heterozygotesi and wild type littermates. No significant difference was found between wild type and RARβ null lung explants cultured for 24 hours without retinoid treatment (data not shown). The explants were then treated for 48 hours with 10⁻⁶ M RA (FIG. 3C). Both wild type and RARβ^{+/-} RA-treated explants exhibited a significant reduction in ATBN, when compared to controls (i.e. RARβ^{+/-} explants cultured with ethanol alone). In contrast, there was no significant difference in ATBN between RA-treated RARβ null explants and controls (FIG. 3C). These data indicate that RARβ is essential for RA-induced inhibition of branching.

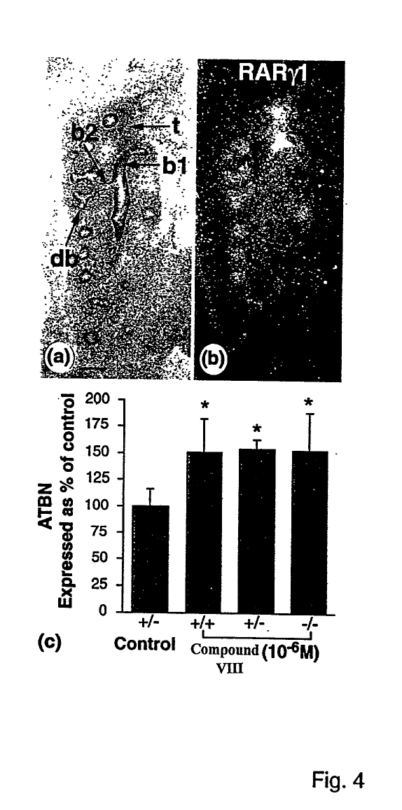

RARα1, RARα2 and RARγ2 transcripts are expressed ubiquitously in E13.5 lungs in vivo. Thus, aside from RARβ isoforms (Ghyselinck, N. B., et al., Dev. Biol. 198:303-318 (1998)), RARγ1 is the only RAR isoform displaying a restricted pattern of expression in the developing lung, being expressed preferentially within the distal bud epithelium (FIGS. 4A and 4B). In order to investigate whether RARγ could be involved in the RA-induced inhibition of lung branching, RARγ null lungs (Lohnes, D., et al., Cell 73:643-658 (1993)) were cultured in the presence of the pan-RAR antagonist. Wild type, RARγ^{+/-} and RARγ null lungs treated with 10⁻⁶M Compound VIII all responded with a similar increase in bud formation when compared to ethanol-treated RARγ^{+/-} controls (FIG. 4C). Thus, RARγ is clearly not required in transducing the RAR antagonist signal which augments distal lung bud formation.

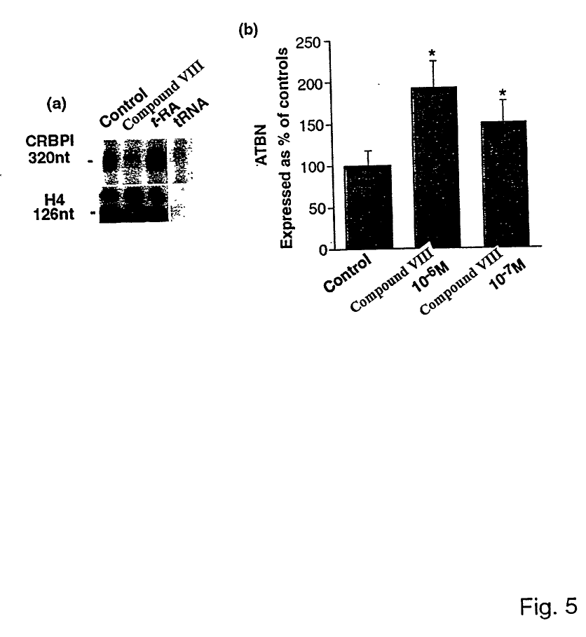

The lung at the pseudoglandular stage is a major site of expression of CRBPI in the embryo (Dollé, P., et al., Development 110:1133-1151 (1990)). In cultured E12.5 wild type lungs, CRBPI expression was increased in the presence of 10⁻⁶ M RA and decreased by 10⁻⁶ M Compound VIII (FIG. 5A). Thus, increased and decreased expression of CRBPI are correlated with retinoid-induced inhibition and stimulation of branching, respectively. To further test an involvement of CRBPI in lung morphogenesis, explants from E12.5 CRBPI null mutants (Ghyselinck, N. B., et al., EMBO J. 18:4903-4914 (1999)) were cultured in the presence of various concentrations of the pan-RAR antagonist. In CRBPI null lungs, but not wild type lungs, a concentration of Compound VIII as low as 10⁻⁷ M induced a significant increase in ATBN (FIG. 5B), indicating that CRBPI null lungs displayed a higher sensitivity to RAR antagonism.

Discussion

Recent work has provided evidence that RA and its nuclear receptors are instrumental for alveolar septation. Administration of RA to newborn rats increases the number of alveoli and restores alveolar number in animal models of emphysema (Massaro, G. D. and Massaro, D., Am. J. Physiol. 270: L305-L310 (1996); Massaro, G. D., and Massaro, D., Nat. Med. 3:675-677 (1997)). Alveolar septation is a late developmental event, as it is initiated only at the end of the fetal period, and essentially takes place during early post-natal life in rodents (reviewed in Hogan, B. L. and Yingling, J. M., Curr. Opin. Genet. Dev. 8:481-486 (1998)). In the present study, we have analyzed the role of RA during the embryonic and pseudoglandular stages of prenatal lung development.

The inhibition of primary lung bud and oesophagotracheal fold formation induced in cultured embryos by the pan-RAR antagonist and observed at a stage equivalent to E9.5 in vivo indicates that RA is normally required for the appearance of these structures. This observation also indicates that the severe lung hypoplasia (or agenesis) and absence of the oesophagotracheal septum previously described at fetal stages in retinoic acid receptor compound mutant mice, as well as in VAD rats, are determined prior and/or during the embryonic stage of lung development. In keeping with this idea, a recent analysis of RARα^{-/-}/RARβ^{-/-} embryos shows that the left primitive lung bud and left oesophagotracheal fold are markedly hypoplastic or absent at E9.5, i.e., at the earliest developmental stage when the primary lung buds and tracheal diverticulum can be identified morphologically. The genetic dissection of the retinoid signaling pathway disclosed herein strongly suggests that the functional heterodimers involved in the primary lung and tracheal bud formation are RARα/RXRα (Kastner, P., et al, Development 124:313-326 (1997); Mascrez, B., et al., Development 125:4691-4707 (1998)).

At the pseudoglandular stage of lung development, RARβ is preferentially expressed in the proximal clefts of the pulmonary tree (Dollé, P., et al., Development 110:1133-1151 (1990); Ghyselinck, N. B., et al., Dev. Biol. 198:303-318 (1998)). Proximal clefts correspond to areas of morphogenetic stability when compared to the distal tips of the pulmonary tree which are the major sites of budding (Hilfer, S. R., et al., Tissue Cell 17:523-538 (1985); Mollard, R. and Dziadek, M., Am. J. Respir. Cell Mol. Biol. 19:71-82 (1998)). The present data demonstrate that, during the pseudoglandular stage of lung development, a block in RA signaling increases formation of distal buds in wild type lungs and that RA inhibits branching in wild type lungs but not in RARβ null lungs. Moreover, the branching inhibition induced by RA treatment is correlated with ectopic expression of RARβ in distal buds, whereas the increase in distal bud number caused by a block in RA signaling is correlated with a decrease of RARβ expression in the pulmonary tree. Collectively, these findings provide evidence that activation of RARβ by RA favors morphogenetic stabilization over de novo budding during formation of the pulmonary tree.

At the pseudoglandular stage of lung development, the RARγ1 isoform is preferentially expressed in the distal buds. The findings that (i) RARγ null lungs respond to a block in RA signaling similarly to wild type lungs, and (ii) that RARβ null lungs, which still express RARγ, are refractory to RA-induced branching inhibition altogether suggest that RARγ is dispensable for the transduction of RA-mediated patterning cues during lung branching.

It has been proposed that CRBPI could play a role in RA synthesis (Napoli, J. L., Biochim. Biophys. Acta 1440:139-162 (1999)). The observation that CRBPI null lungs are approximately 10-fold more sensitive than wild type lungs to the stimulatory effect of the pan-RAR antagonist upon distal bud formation suggests indeed that the CRBPI present in the lung could be involved in the production of RA in this developing organ. Thus, it is conceivable that the actual level of CRBPI in the lung at the pseudoglandular stage could participate in the control of branching morphogenesis.

The present study demonstrates that RA is essential at two stages of prenatal lung development. First, an RA signal transduced through RARα/RXRα heterodimers is required for the evagination of the primary lung bud from the primitive foregut. Subsequently, RA signaling through RARβ inhibits lung budding thereby specifying the morphogenetically inactive regions which form conducting airways.

Experimental Procedures

1. Lung and Whole Embryo Culture

The mouse lines carrying the RARβ, RARγ and CRBPI null mutations and their genotyping protocols have been described previously (Lohnes, D., et al., Cell 73:643-658 (1993); Ghyselinck, N. B., et al., Int. J. Dev. Biol. 41:425-447 (1997); Ghyselinck, N. B., et al., EMBO J. 18:4903-4914 (1999)). The morning of appearance of the vaginal plug was designated as E0.5. For explant culture, E11.75 and E12.5 lungs from RARβ^{+/-}×RARβ^{+/-}, RARγ^{+/-}×RARγ^{+/-}, CRBPI^{-/-}×CRBPI^{-/-} and wildtype crosses were dissected in PBS and incubated in BGJB medium (Fitton-Jackson modified, GibcoBRL), 5% delipidated fetal calf serum (FCS, GibcoBRL) and 180mg/ml vitamin C (Sigma) on millipore filters (GibcoBRL), at 37° C., in the presence of 5% CO₂. Half of the media, supplemented with fresh retinoids (see below), was changed daily. The cultured lung buds reproducibly grew and branched for at least seven days in culture (data not shown). Average terminal bud number (ATBN) was calculated after counting every bud in each explant at 0, 24, 48, 72 and 96 hours after the commencement of culture. Significant differences in ATBN between different groups were determined by ANOVA and Newman-Keuls multiple comparison tests according to previously described methods (Motulsky, H., Intuitive Biostatistics, New York: Oxford University Press (1995)). The synthetic retinoid Compound VIII [a specific pan RAR (α, β and γ) antagonist (Bristol-Myers-Squibb, N.J.); Chazaud, C., et al., Development 126:2589-2596 (1999); Wendling, O., et al., Development 127:1553-62 (2000)] and RA (all-trans retinoic acid) (Sigma) were diluted in ethanol and added to the culture medium at a final ethanol concentration of 0.1% at the beginning of culture and every subsequent 24 hours.

Whole embryos from CD 1×CD 1 crosses were collected at E8.0, staged and cultured according to previously described methods in the presence of retinoids or ethanol vehicle alone for 48 hours (New D.A.T., Postimplantation Mammalian Embryos: A Practical Approach, Cop, A. J. and Cockroft, D. L., eds., Oxford University Press (1990); Wendling, O., et al., Development 127:1553-62 (2000)). For morphological assessment, serial transverse histological sections were stained with hematoxylin and eosin.

2. RNAse Protection and In Situ Hybridization

Total RNA preparation, RNAse protection assays and in situ hybridizations were performed as previously described (Chirgwin, J. M., et al., Biochemistry 18:5294-5299 (1979); Décimo, D., et al., “In situ hybridization of nucleic acid probes to cellular RNA,” in Gene Probes 2, A Practical Approach, Hames, B. D. and Higgins, S., eds.: Oxford University Press (1995) pp. 183-210; Mollard, R. and Dziadek, M., Int. J. Dev. Biol. 41:655-666 (1997); Ghyselinck, N. B., et al., Int. J. Dev. Biol. 41:425-447 (1997)). CRBPI, H4, RARβ and vimentin cDNAs have been previously described (Ghyselinck, N. B., et al., Int. J. Dev. Biol. 41:425-447 (1997); Mollard, R. and Dziadek, M., Int. J. Dev. Biol. 41:655-666 (1997)).

All documents, e.g., scientific publications, patents and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method of treating lung branching malformation in a subject in need thereof, said method comprising administering to said subject a pharmaceutically effective amount of a retinoic acid receptor (RAR) retinoid.

2. The method of claim 1, wherein said malformation is due to decreased lung branching.

3. The method of claim 2, wherein said malformation is lung hypoplasia.

4. The method of claim 2, wherein said method further comprises administering to said subject a pharmaceutically effective amount of a cellular retinol binding protein-I (CRBPI) antagonist.

5. The method of claim 4, wherein said CRBPI antagonist is selected from the group consisting of a CRBPI antibody, a CRBPI antisense oligonucleotide, a RAR antagonist, and dibutyryl cAMP.

6. The method of claim 2, wherein said RAR retinoid is an antagonist.

7. The method of claim 6, wherein said antagonist is an RARβ antagonist.

8. The method of claim 1, wherein said malformation is due to increased lung branching.

9. The method of claim 8, wherein said malformation is lung hyperplasia.

10. The method of claim 8, wherein said method further comprises administering to said subject a pharmaceutically effective amount of a cellular retinol binding protein-I (CRBPI) agonist.

11. The method of claim 10, wherein said CRBPI agonist is a retinoic acid receptor (RAR) agonist.

12. The method of claim 10, wherein said CRBPI agonist is selected from the group consisting of dexamethasone, triiodothyronine, and transforming growth factor β.

13. The method of claim 8, wherein said RAR retinoid is an agonist.

14. The method of claim 13, wherein said agonist is an RARβ agonist.

15. A method of increasing alveoli in a subject in need thereof, said method comprising administering to said subject a pharmaceutically effective amount of a retinoic acid receptor (RAR) antagonist.

16. The method of claim 15, wherein said method further comprises administering to said subject a pharmaceutically effective amount of a cellular retinol binding protein-1 (CRBPI) antagonist.

17. The method of claim 16, wherein said CRBPI antagonist is selected from the group consisting of a CRBPI antibody, a CRBPI antisense oligonucleotide, a RAR antagonist, and dibutyryl cAMP.

18. The method of claim 15, wherein said subject suffers from a disease selected from the group consisting of: chronic obstructive pulmonary disease, emphysema, chronic bronchitis, interstitial fibrosis, pulmonary tuberculosis, and sarcoidosis.

19. The method of claim 15, wherein said RAR antagonist is an RARβ antagonist.

20. A method of inducing primary lung bud formation in a subject, said method comprising administering a pharmaceutically effective amount of a retinoic acid receptor (RAR) agonist.

21. A method of identifying an agent capable of inducing primary lung bud formation, said method comprising:
(a) administering an agent to an embryo; and
(b) determining primary lung bud formation of said embryo.

22. The method of claim 21, further comprising:
(c) comparing (b) with an embryo untreated with said agent.
